(19) United States
(12) Patent Application Publication
(10) Pub. No.: US 2008/0086118 A1
(43) Pub. Date: Apr. 10, 2008

(54) Apparatus and method for diode-pumped laser ablation of soft tissue

(75) Inventors: Ming Lai, Rochester, NY (US); Liyue Mu, Fremont, CA (US); Kangze Cai, Fremont, CA (US); Weiguo Luo, Fremont, CA (US)

Correspondence Address: PILLSBURY WINTHROP SHAW PITTMAN, LLP, P.O. BOX 10500, MCLEAN, VA, 22102, US

(73) Assignee: Applied Harmonics Corporation, Fremont, CA (US)

(21) Appl. No.: 11/804,556

(22) Filed: May 17, 2007

Related U.S. Application Data
(60) Provisional application No. 60/801,149, filed on May 17, 2006.

(51) Int. Cl. A61B 18/20

(52) U.S. Cl. 606/11

(57) ABSTRACT

The present invention fulfils a need to advance a diode-pumped solid-state laser (DPSSL) and to employ laser-induced thermal explosive ablation (LITEA) for prostate surgery. The present invention operates a quasi-CW Q-switched DPSSL at a lower repetition rate of 5 kHz to 20 kHz to produce bigger pulse energy of 2.5 mJ to 10 mJ, while maintaining optimal average power. The present invention also employs two AO Q-switches or one EO Q-switch to obtain shorter pulse duration of 40 ns to 150 ns and to obtain higher peak power of 50 kW to 100 kW, leading to a pulse fluence of 250 mJ/cm² to 1000 mJ/cm² on target tissues. The present invention further applies electronic control to suppress amplitude fluctuation below 5% to ensure smoother ablation profile.

### REFERENCE CITED

U.S. Pat. No. 6,986,764B2 January 2006 Davenport, et al

U.S. Pat. No. 5,312,396 May 1994 Feld, et al

U.S. Pat. No. 6,413,267B1 July 2002 Dumoulin-White, et al

U.S. Pat. No. 6,554,824B2 April 2003 Davenport, et al.

U.S. Pat. No. 6,309,352B1 October2001 Oraevsky, et al.

U.S. Patent Application Publication 2004/0236319 A1 Davenport, et al.

U.S. Patent Application Publication 2005/0256513 A1 Murray, et al

U.S. Patent Application Publication 2005/0027286 A1 Davenport, et al

U.S. Patent Application Publication 2005/00288653 A1 Lai, et al

### OTHER PUBLICATIONS

Tuan Vo-Dinh, “Biomedical Photonics Handbook”, page 2-1 to 2-75, 5-1 to 5-16, CRC Press, 2003

V. V. Golovlev et al, “Ablation of an optically homogeneous absorbing medium by scattered pulsed laser radiation”, Appl. Phys. B57, 451, 1993

R. O. Esenaliev et al, “Laser ablation of aqueous solutions with spatially homogeneous and heterogeneous absorption”, Appl. Phys. B59, 73, 1994

Lawrence Livermore National Lab, “The Short-Pulse Laser: A Safe, Painless Surgical Tool” Science & Technology Review, October 1995

A. Vogel and V. Venugopalan, “Mechanisms of Pulsed Laser Ablation of Biological Tissues”, Chem. Rev. 103, 577-644, 2003

V. Venugopalan et al, “Thermodynamic Response of Soft Biological Tissues to Pulsed Infrared-Laser Irradiation”, Biophysical Journal, Vol. 70, 2981-2993, 1996

M. Ogura et al, “Myocardium Tissue Ablation With High-Peak-Power Nanosecond 1,064- and 532-nm Pulsed Lasers: Influence of Laser-Induced Plasma”, Lasers in Surgery and Medicine, 31:136-141, 2002

J. Niamtu, “Clinical Applications of the 532-nm Diode Laser for the Treatment of Facial Telangiectasia and Pigmented Lesions: Literature Review, History, and Discussion of Clinical Experience” The American Journal of Cosmetic Surgery Vol. 18, No. 2, 2001

S. Uhlhorn, “Free Electron Laser Ablation of Soft Tissue: The Effects of Chromophore and Pulse Characteristics on Ablation Mechanics” Ph.D. Dissertation, Vanderbilt University, 2002

A. F. El-Sherif and T. A. King, “Soft and hard tissue ablation with short-pulse high peak power and continuous thulium-silica fiber lasers”, Lasers Med Sci.; 18(3):139-47, 2003

A. Liu et al, “60-W green output by frequency doubling of a polarized Yb-doped fiber laser”, Optics Letters, Vol. 30, No. 1, 2005

F. H. Loesel et al “Laser-induced optical breakdown on hard and soft tissues and its dependence on the pulse duration: experiment and model”, IEEE Journal of Quantum Electronics, Vol. 32, 10, 1717-1722, 1996

F. Sengor et al, “A comparative study of laser ablation and transurethral electroresection for benign prostatic hyperplasia: results of a 6-month follow-up”, British Journal of Urology, 78, 398-400, 1996

Malek et al., “High-Power Potassium-Titanyl-Phosphate (KTP/532) Laser Vaporization Prostatectomy: 24 Hours Later”, Urology 51: 254-256, 1998, Elsevier Science Inc.

R. S. Kuntzman et al, “High-Power Potassium Titanyl Phosphate Laser Vaporization Prostatectomy”, Mayo Clin Proc. 73:798-801, 1998

R. S. Malek et al, “High Power Potassium-Titanyl-Phosphate Laser Vaporization Prostatectomy”, J. of Urology, Vol. 163, 1730-1733, 2000

M. A. Hai et al, “Photoselective Vaporization of the Prostate: Initial Experience with a New 80 W KTP Laser for the Treatment of Benign Prostatic Hyperplasia”, J. of Endorology, Vol. 17, (2), 2003.

M. Grasso et al, “Lasers in Urology”, http://www.emedicine.com/med/topic3037.htm, March 2006

A. E. Siegman, “Lasers”, University Science Books, California, 1986

This application claims the benefit of U.S. Provisional Application No. 60/801,149, filed on May 17, 2006.

### FIELD OF THE INVENTION

This invention relates in general to laser ablation of human soft tissue and in particular to laser-induced thermal explosive ablation of prostate tissue.

### BACKGROUND

Laser-induced thermal explosive ablation (LITEA) is a well-known and well-understood process, as described by Vogel and Venugopalan in Mechanisms of Pulsed Laser Ablation of Biological Tissues, which is incorporated by reference. Laser ablation occurs rapidly and explosively when a pulsed laser of short pulse width and high fluence impinges onto a biological tissue. One character of LITEA is its explosive tissue removal as a good percentage of the laser energy is converted into kinetic energy of tissue debris. Another character of LITEA is a threshold in pulse duration and pulse fluence, below which other ablation mechanism may dominate. A further character of LITEA is its minimal thermal damage of neighboring tissue. LITEA is thus an ideal surgical procedure for large volume removal of soft tissue, such as laser ablation of prostate tissues.

Diode-pumped solid-state laser (DPSSL) has many advantageous features over its ancestor lamp-pumped solid-state laser. DPSSL has substantially higher efficiency to convert electric power to laser output, longer lifetime to minimize service and maintenance cost, and compact package to render user-friendly and convenience. DPSSL has become popular in many medical and surgical laser systems. Therefore, it is greatly desirable to implement DPSSL for prostate surgery.

In the past decade, laser prostate surgery has become an alternative option to treat Benign Prostatic Hyperplasia (BPH), which troubles elderly men with symptoms such as urinary frequency, dysuria and incomplete bladder emptying. High power laser beam is delivered to target prostate tissue through an optical fiber that is introduced through an endoscope or cystoscope. Surgical outcome of laser treatment depends on a number of factors, including wavelength, power density, pulse duration, and pulse fluence.

High average power (60-80 W) Nd:YAG laser with a wavelength of 1064 nm was first used for BPH treatment in early 1990s. The laser-irradiated tissue is heated up to boiling temperature, the top layer is thus evaporated and the under layer is coagulated. The advantage of Nd:YAG laser surgery is its excellent hemostatic effect, due to a 7 mm penetration depth and thus a thick layer of tissue coagulation. However, the Nd:YAG laser treatment of obstructive BPH in general is not as effective as transurethral resection of the prostate (TURP), the surgical “gold standard” for decades.

High power (60-100 W) Ho:YAG laser with a wavelength of 2140 nm can be strongly absorbed by water and can thus evaporate soft tissue effectively. Ho:YAG laser surgery is a transurethral procedure and its clinical outcome is comparable with TURP. However, Ho:YAG laser surgery is technically challenging and has a steep learning curve.

A 60 W average power, Q-switched and frequency-doubled Nd:YAG laser was reported to conduct BPH treatment in 1998, by Malek et al. This laser is lamp pumped to produce quasi-CW Q-switched pulses at 532 nm, which is transparent in water but selectively absorbed by oxyhemoglobin in virgin soft tissue. This KTP/532 laser at 60 W can effectively vaporize and ablate oxyhemoglobin-concentrated prostate tissue and concurrently achieve some level of hemostasis. The surgical outcome is comparable with TURP while the complication is significantly reduced. However, the laser efficiency is less ideal and the length of the procedure needs improvement.

In U.S. Pat. Nos. 6,554,824 and 6,986,764, Davenport et al disclose an improvement of the above KTP/532 laser by operating the laser in a ‘macro-pulsed’ mode, by increasing the average power to 80 W, by decreasing laser beam M² value to less than 144, and by creating higher power density on the tissues. Macro-pulsed mode is an enabling element of the improvement, which leads to a significantly higher conversion efficiency for green laser generation. This significantly improved laser efficiency results in a higher laser output of 80 W and meanwhile a better beam quality, which enables a higher power density on tissue and a faster laser ablation. The disclosed ablation procedure, named photoselective vaporization of the prostate (PVP), is thus associated with many benefits of macro-pulsed operation of lamp-pumped KTP/532 laser.

Macro-pulsed operation of a high power laser is, however, found unpractical with diode-pumped solid-state lasers, which are more preferable for surgical systems over lamp-pumped lasers. Macro-pulsed operation of a high power DPSSL requires macro-pulsed operation of high power diode lasers, which results in reduced lifetime and drifting wavelength of the diode laser source. Besides, the KTP/532 laser has pulse duration of 450 ns, which is too long for an efficient LITEA.

To implement DPSSL for prostate surgery and to employ LITEA for rapid and explosive ablation, a quasi-CW Q-switched DPSSL is developed to deliver laser pulses of 150 ns pulse duration and 10 mJ pulse energy at green wavelength. The laser can produce on prostate tissue a peak power of 60 kW and a pulsed fluence of 1000 mJ/cm². A comparison experiment shows that the DPSSL demonstrates higher ablation efficiency over a PVP laser of the same average power.

### THEORY OF LASER-INDUCED THERMAL EXPLOSIVE ABLATION

In general, laser tissue ablation always depends on the wavelength of laser radiation. The absorbing chromophores in the tissue are either proteins or water. In either case, water serves as a reservoir for the thermal energy into which the laser-radiation energy is converted. When a light beam travels after a certain distance in a biological tissue, the optical transmission T is governed by Beer-Lambert's law:

$$T = \frac{I}{{I}_{0}} = {ⅇ}^{- {μ}_{a} l}$$

Where I₀ is the laser light intensity at the tissue surface, I is the laser light intensity at a distance l below the surface, and μₐ is tissue optical absorption coefficient. The value of μₐ varies for different tissues, tissue structures, and laser wavelengths. The higher μₐ is, the shallower a light beam can penetrate through the tissue. For laser-tissue ablation, it always chooses a laser wavelength that can be highly absorbed by the tissue or water.

Peak power of a laser pulse is calculated by dividing laser pulse energy by pulse width. High pulse energy and short pulse width result in high peak power. Laser pulse peak power and laser average output power are two different concepts. For explosive thermal ablation of biological tissues, laser pulse peak power plays an important role. When laser pulse is short enough, such as sub 150 ns, substantial tissue ablation happens after the completion of laser pulse irradiation.

When pulsed laser with short pulse width and high pulse energy irradiates on biological tissues, two important photophysical phenomena take place, i.e. thermal confinement and stress confinement. They are necessary conditions for effective thermal explosive ablation. The thermal confinement is spatially confined microsurgical effects, which can be achieved by the use of laser exposures that are shorter than the characteristic thermal diffusion time of the heated volume. For laser tissue ablation, the heated volume is typically a layer of tissue that has a thickness of 1/μₐ, and the characteristic thermal diffusion time, t_{d}, is given by:

$${t}_{d} = \frac{1}{k {μ}_{a}^{2}}$$

where k is the thermal diffusivity. When laser exposure is shorter than the characteristic thermal diffusion time of the heated volume, or laser pulse width tₚ<t_{d}, the heat is confined inside the laser irradiated volume. The thermal confinement condition is satisfied.

Pulsed laser ablation also generates thermoelastic stresses. The stress wave moves at speed of sound inside tissue. If the laser pulse duration tₚ is shorter than or on the order of the characteristic time for a stress wave to propagate across a distance of 1/μₐ in the heated volume, the condition of “stress confinement” is met. That is:

$${t}_{p} \leq \frac{1}{{c}_{a} {μ}_{a}}$$

where cₐ is the speed of sound in tissue. In this case, heating of the irradiated volume is achieved under isochoric conditions, and the internal stresses generated do not propagate outside the heated volume during the laser irradiation. In stress-confined region, strong heating leads to a substantial rise of pulsed pressure. Short laser pulses may also initial break down of tissue structure, because the tensile component of thermoelastic stresses enhances bubble formation and vaporization, which may contribute to fracture of the tissue structure.

A key parameter of thermal explosive ablation with short laser pulses is an ablation threshold of laser pulse fluence, measured by irradiated laser energy per square centimeters. Actual ablation threshold depends on many physical properties of the tissue, including optical absorption coefficient μₐ, thermal diffusivity k and speed of sound cₐ inside tissue, as well as laser pulse duration. Lower ablation threshold is usually associated with higher ablation efficiency. Because of significant thermal confinement and stress confinement, short pulse laser leads to a reduction of the threshold for ablation. Many studies demonstrate that stress confinement can reduce ablation threshold by its catalytic effect on bubble nucleation and explosive boiling. For given pulse energy, the shorter the pulse width, the lower the ablation threshold. The peak stress of a compressive recoil stress σₚ, which plays an important role to explode tissues, is reversely proportional to laser pulse width tₚ:

$${σ}_{p} ∝ \frac{1}{{t}_{p}}$$

Since short pulse width increases the peak stress, Venugopalan et al conclude that the shorter is the laser pulse width the lower is the volumetric power density or explosive ablation threshold required for generating a phase explosion. The mass removal rate m is given by:

$$m = \frac{ρ}{{μ}_{a}} ln ( \frac{{μ}_{a} {ɛ}_{0}}{{μ}_{a} {ɛ}_{th}} )$$

where m is removed mass per unit area, ρ is tissue mass density, μₐ is tissue optical absorption coefficient, μₐε₀ is incident laser volumetric energy density per unit area on the tissue surface, and μₐεₜₕ is threshold energy volumetric density per unit area required for ablation. For fresh prostate tissue, in which hemoglobin has rich concentration, the μₐ approximately equals to tμ_{a(hemoglobin)}. For coagulated prostate tissue, however, hemoglobin has very limited concentration, the μₐ approximately equals to μ_{a(coagulated prostate tissue)}. For a given tissue, a decrease in ablation threshold of pulse fluence results in an increase in mass removal efficiency. Since a short laser pulse leads to lower ε_{E th}, it ablates tissues more efficiently than a long laser pulse does.

At a wavelength shorter than 1400 nm, laser radiation can be absorbed by microchomophore centers in the tissue, in which volume of microchomophore centers is materially smaller than the total volume of irradiated tissue. When thermal confinement and stress confinement are satisfied under high peak power pulsed laser irradiation, these overheated microchomophore centers go to explosive boiling, mechanically removing the entire irradiated volume. Evidence shows that only 10% of the ablated tissue suffers evaporation even at the ablation threshold; the rest of the ejected mass is in the form of micro particles. At visible wavelength, the microchomophore center is hemoglobin when prostate tissue is fresh; it is the coagulated tissue when prostate is coagulated.

To achieve precise tissue ablation, minimizing thermal damage of neighbor tissue is an ultimate goal of laser surgeries. Precise tissue ablation requires the use of laser wavelengths having a small optical penetration depth in tissue such that energy deposition is confined to a small volume. Thermal confinement is required for precise ablation in order to limit the spatial extent of thermal diffusion during irradiation and to maximize the temperatures in the absorbed volume. Additionally, stress confinement provides for a more efficient ablation process as it serves to reduce the volumetric energy density required for material removal. Thermal confinement and stress conferment together result in effective and precise tissue ablation and reduced thermal damage of adjacent tissues.

Controlling thermal side effects is also important in laser surgery. Using short laser pulses to minimize heat diffusion into adjacent tissue, laser thermal side effects can be reduced. Thermal side effects can be diminished by further reducing laser pulse durations to provide both stress and thermal confinement, because lowering the ablation enthalpy in the stress confinement regime reduces the residual heat in the tissue.

In the process of laser-induced thermal explosive ablation, tissue carbonization is negligible. Tissue carbonization is typically resulted from the use of long exposure times or low peak power pulses wherein tissues are heated up to a temperature about 150° C. In contrast, laser pulses with short pulse width and high peak power quickly increases the tissue temperature to close to 400° C., and explosive ablation dominates the laser-tissue interaction.

In addition to laser-induced thermal explosive ablation at visible spectrum, high peak power UV laser pulses and high peak power near-infrared laser pulses can also effectively and explosively ablate soft tissues. At deep UV, where laser wavelength is shorter than 250 nm, laser-tissue interaction is dominated by photochemical reactions. In a peak power density above 10 MW/cm², deep UV laser pulses may produce a thermal explosive ablation similar to that of visible laser pulses. At a peak power density of about 800 MW/cm², Ogura et al have demonstrated that laser pulses at 1064 nm near infrared ablate soft tissues via a photo-disruption process and achieve ablation efficiency better than laser pulses at 532 nm.

### SUMMARY OF THE INVENTION

The present invention recognizes a need to advance a diode-pumped solid-state laser (DPSSL) and to employ laser-induced thermal explosive ablation (LITEA) for prostate surgery. The present invention contemplates to operate a quasi-CW Q-switched DPSSL at a lower repetition rate of 5 kHz to 20 kHz to produce bigger pulse energy of 2.5 mJ to 10 mJ, while maintaining optimal average power. The present invention also contemplates to employ two AO Q-switches or one EO Q-switch to obtain shorter pulse duration of 40 ns to 150 ns and to obtain higher peak power of 50 kW to 100 kW, leading to a pulse fluence of 250 mJ/cm² to 1000 mJ/cm² on target tissues. The present invention further contemplates to apply electronic control to suppress amplitude fluctuation below 5% to ensure smoother ablation profile.

LITEA is to ablate biological soft tissues under thermal confinement and stress-confinement conditions and to achieve an explosive character of ablation. When laser pulse duration is shorter than 150 nanoseconds, laser energy can be more effectively converted into mechanical energy. With such a short pulse, bubble formation starts at the end of laser irradiation. This leads to extremely high volumetric energy densities and temperatures and thus a superheated condition. Laser pulse peak power density can reach 30 MW/cm² on the irradiated area, and volumetric power density may be over 300 MW/cm³ inside coagulated tissues. As a result, the cavitation bubble expands to a larger size than those produced by 450 ns laser pulses in the prior art. In LITEA with sub-150 ns pulses, the entire pulse energy is absorbed before significant material removal occurs, and thus the tissue volume that reaches a threshold volumetric energy density is removed explosively.

By employing LITEA, improved ablation efficiency can be obtained without boosting laser average output power, which is usually the major cost for the already expensive laser surgical systems. An additional benefit of LITEA is less thermal damage to the surrounding tissues.

For biological tissue, the longer the wavelength is, the deeper is the laser penetration. Soft tissue, such as prostate tissue, has limited optical absorption at both visible and near infrared wavelength if there is no hemoglobin or blood contained. Due to thermal denaturation, coagulated prostate tissues may have rather different optical properties from virgin tissues. Optical absorption coefficient of coagulated prostate tissues may increase to a few cm⁻¹ at near infrared wavelength and be much larger at shorter wavelengths. Visible wavelength is preferable for LITEA because coagulated prostate can more effectively absorb visible light than near infrared light. Good absorption at visible wavelength directly results in shallower absorption depth, higher volumetric peak power density, and less thermal damage to adjacent tissues.

In LITEA, tissue removal significantly dominates tissue coagulation. This is in contrast with laser ablation with long pulses. On the other hand, appropriate tissue thermal coagulation helps for hemostasis. In one embodiment of the present, near-infrared laser of mid-low power is also employed to obtain soft tissue coagulation. Such a dual-wavelength laser system takes the advantages of an ablative laser for its efficient explosive ablation and a coagulative laser for hemostasis. A dual-wavelength laser system can be delivered, for instance, from an Nd:YAG laser producing high peak power at green wavelength and low peak power at near-infrared wavelength. Near-infrared laser power shall not be too high to avoid deep tissue coagulation and carbonization, which can induce complications such as dysuria.

In another embodiment, the laser pulses are shortened to less than 150 nanoseconds at 10 kHz during normal operation. In case of bleeding during the surgery, the pulse repetition rate can be increased to 30 kHz or higher to lengthen the pulse width to a few hundred nanoseconds and to drop the peak power density on the target tissue. This way, laser coagulation is enhanced to improve hemostasis.

For a continuously pumped, Q-switched solid-state laser, the laser pulse width tₚ can be given approximately by

$${t}_{p} \approx \frac{r η ( r )}{r - 1 - ln r} {τ}_{c}$$

Where r is initial inversion ratio determined by the pumping power, Q-switching repetition rate, laser rod diameter and laser cavity design; η(r) is energy extraction efficiency determined by initial inversion ratio r and Q-switch type; τ_{c} is the laser cavity decay time determined by laser cavity round-trip time, or cavity length, and type of laser gain medium. To obtain sub-150 ns pulse width, in one of the embodiments, the present invention chooses a laser gain medium of Nd:YAG or Nd:YVO₄ for longer upstate lifetime, short cavity length for short cavity decay time, small diameter of laser rod for higher single pass gain, and fast Q-switch for small energy extraction efficiency while keeping high pulse energy. This invention also electronically optimizes Q-switch duty circle, energy extraction, and repetition rate to obtain shorter laser pulse width and higher pulse energy. The achievable pulse width at visible wavelength can be much shorter than 150 ns. The laser pulses obtained are capable to produce LITEA on varieties of soft tissues.

High peak power may cause optical damage of laser optics. The present invention operates the high power laser at quasi-CW Q-switching mode and highly multiple transverse modes to reduce intracavity peak power density and thus to avoid power damage of laser optics.

The present invention also elaborates to reduce beam divergence and beam spot size on target tissue to ensure LITEA. The present invention further minimizes power consumption of the laser system such that external water-cooling or secondary cooling loop can be eliminated. The frequency-doubling crystal is positioned inside a temperature-regulated housing. Phase-matching angle of the crystal is adjusted through temperature tuning to avoid crystal damage due to temperature shock, which may otherwise occur when phase-matching angle is tuned mechanically.

In a first embodiment of the present invention, a diode-pumped Nd:YAG laser is developed to employ laser-induced thermal explosive ablation (LITEA) of prostate tissues. The laser cavity length is less than 60 cm for short cavity decay time. Two AO Q-switches with fast rise time are synchronized and located on each side of Nd:YAG rod to obtain higher Q switching. Modulation depth of an AO Q-switch is polarization dependent, and AO scattering directions of the two Q-switches are oriented perpendicular to each other. The Nd:YAG rod has a diameter of 5 mm or smaller for higher single pass gain. The diode-pumped laser is operated at a continuous Q-switching mode. The laser is frequency-doubled with an LBO crystal to produce laser output at 532 nm. In a normal operation, this embodiment generates laser pulses with sub-150 ns duration at a repetition rate of 15 kHz or lower. The controlling electronics optimizes pump power and repetition rate to achieve about 1000 mJ/cm² pulse fluence on target tissues. When it is needed to improve hemostasis, controlling electronics lengthens the pulse duration to enhance tissue coagulation.

In a second embodiment, the diode-pumped laser employs two Nd:YAG rods in cascade to increase single pass gain. Each Nd:YAG rod has a diameter of 4 mm or less. Due to a higher initial inversion ratio, this embodiment generates laser pulses with sub-50 ns duration at a repetition rate of 15 kHz or lower.

In a third embodiment, the diode-pumped laser employs an EO Q-switch to achieve faster Q-switching time. Nd:YAG or Nd:YVO₄ laser rod is used. A Brewster angel polarizer and a quarter-wave plate are implemented to make EO Q-switching possible. Because of a deeper modulation of the EO Q-switch, this embodiment generates laser pulses with sub-10 ns duration at a repetition rate up to 10 kHz.

In a fourth embodiment, the diode-pumped laser is implemented with Yb-doped large core fiber. The laser is frequency-doubled with an LBO or KTP crystal to produce green wavelength around 540 nm. The laser is constructed with fiber-oscillator-power-amplifier (FOPA) configuration, in which a low-power pulse oscillator follows by a high-gain, high-power fiber amplifier. The pulse oscillator generates a train of laser pulses with pulse width less than 5 nanoseconds at kilohertz to megahertz repetition rate. The power amplifier may have one or more stages for a desirable output power. This laser may provide close to single mode beam quality with very high wall plug efficiency and without the need of water-cooling. High efficiency frequency doubling can be achieved with such high quality beam to generate high power green laser. This embodiment can generate laser pulses with pulse duration of 1-5 ns and with peak power density more than 10 MW/cm² on target tissues.

In a fifth embodiment, the diode-pumped laser system includes a mid-low power near-infrared laser for tissue coagulation. The coagulation laser can be delivered from a 1064 nm Nd:YAG laser, 1064 nm Nd:YVO4 laser, 800-1000 nm diode laser, 800-1000 nm fiber-coupled diode laser, or 1000-1400 nm fiber laser. For best hemostasis, the coagulation laser produces 5-30 W laser power onto target tissues. This embodiment implements a dual-wavelength laser.

LITEA is more favorable in a liquid environment. For a given radiant exposure, thermal confinement and stress confinement are better enforced in a liquid environment. Consequently, laser energy can be more effectively transferred into disruptive mechanical energy, and ablated mass rate or ablation efficiency is enhanced.

Operating a diode-pumped laser at a continuous Q-switching mode allows continuous operation of the high power diode lasers that provide pump radiation for the solid-state laser. High power diode lasers have typically much longer lifetime in CW mode operation than in pulsed mode operation. Continuous pump and highly multiple-modes operation improve power conversion from electric power to laser power output. Electrical power consumption can be made low enough such that surgical laser systems of this invention can use wall-plug outlet and have no need for external water-cooling or secondary cooling loop. Lower power consumption also reduces operation cost of the surgical laser systems.

Reducing beam divergence and beam spot size on target tissue increases laser power density and thus laser ablation rate. To implement LITEA for prostatectomy, optical fiber is used to deliver laser power to target tissue. Coupling optics is elaborated to reduce beam divergence and to project laser power into a smaller spot than otherwise what a prior art laser system does. A reduced beam divergence leads to a slower power density change with distance between the fiber tip and target tissue, which enables the surgeon to have better control of ablation rate.

In summary, the present invention discloses a surgical system employing diode-pumped laser and LITEA to advance large volume removal of soft tissue. More specifically, a diode-pumped laser delivers green laser pulses of 150 ns pulse width and 1000 mJ/cm² pulse fluence onto target tissues. Dual wavelength or pulse width adjustment is implemented to improve hemostasis.

Accordingly, one objective of the present invention is to provide a new and improved diode-pumped laser to incorporate LITEA for soft tissue ablation.

Another objective of the present invention is to provide a new and improved diode-pumped laser for laser prostatectomy.

The above and other objectives and advantages of the present invention will become more apparent in the following drawings, detailed description, and claims.

### BRIEF DESCRIPTION OF THE FIGURES

FIG. 1 is a schematic diagram showing a first embodiment of a diode-pumped laser system for soft tissue explosive ablation. The laser pulse width is less than 150 ns in normal operation.

FIG. 2 is a schematic diagram showing a second embodiment of a diode-pumped laser system for soft tissue explosive ablation. Two laser rods are used to generate sub-50 ns pulses.

FIG. 3 is a schematic diagram showing a third embodiment of a diode-pumped laser system for soft tissue explosive ablation. EO Q-switching is employed to generate sub-10 ns pulses.

FIG. 4 is a schematic diagram showing a fourth embodiment of a diode-pumped fiber laser system for soft tissue explosive ablation. An amplitude modulator controls pulse width to 5 ns.

FIG. 5 is a schematic diagram showing a fifth embodiment of a dual-wavelength surgical laser system.

FIG. 6 is a schematic diagram showing anther laser beam coupling of the fifth embodiment.

FIG. 7 illustrates photoselective tissue vaporization of prior art. Oxyhemoglobin is the primary chromophore.

FIG. 8 illustrates thermal explosive ablation of fresh tissues. Major absorption chromophores are oxyhemoglobin (HbO₂) and deoxyhemoglobin (Hb). Ablated mass has larger volume than light absorption volume.

FIG. 9 illustrates thermal explosive ablation of coagulated tissues. The primary absorption chromophore is coagulated tissue. Ablated mass has larger volume than light absorption volume.

FIG. 10 illustrates dependence of ablation rate on laser pulse duration. Shorter pulse width enables lower ablation threshold and faster ablation rate.

FIG. 11 illustrates peak power reduction when pulse repetition rate increases.

FIG. 12 illustrates a prior art fiber tip.

FIG. 13 illustrates a modified fiber tip.

FIG. 14 illustrates another prior art fiber tip.

FIG. 15 illustrates another modified fiber tip.

### DETAILED DESCRIPTION OF THE FIGURES

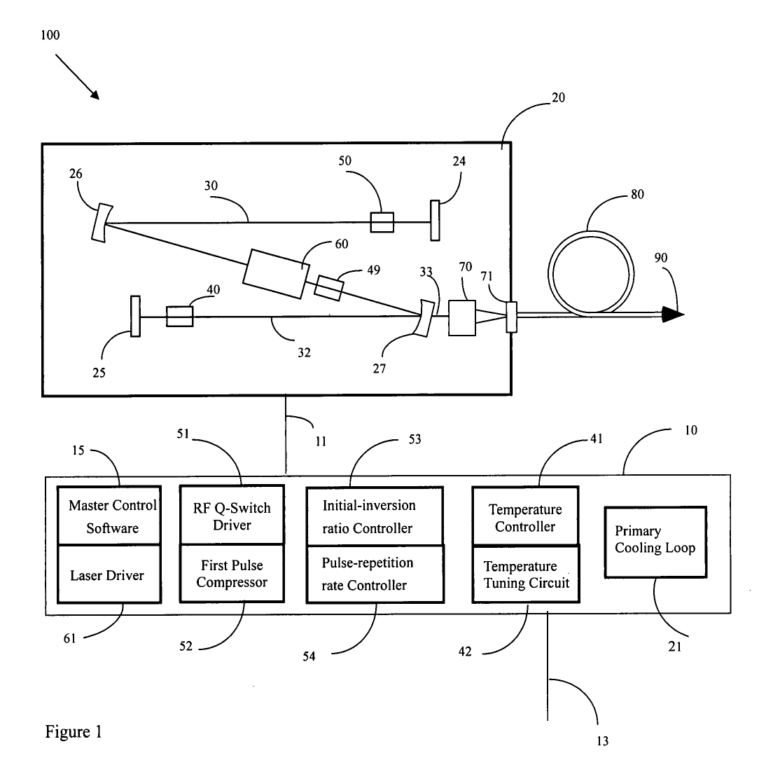

FIG. 1 is a schematic diagram showing a first embodiment of a diode-pumped laser system 100 for soft tissue explosive ablation. The laser system 100 is a diode-pumped Nd:YAG laser that is quasi-CW Q-switched and intracavity-frequency-doubled to produce a laser output 90 of short pulses and high pulse fluence. The laser system 100 includes a laser head 20, a laser controller 10, and an optical fiber 80.

The laser head 20 consists of a pump head 60, two fast rise-time Q-switches 49 and 50, a second harmonic generator 40, and a folded resonant cavity formed by cavity mirror 24, cavity mirror 25, infrared folding mirror 26, and output coupler 27. The cavity mirror 24 and the infrared folding mirror 26 have high reflection coatings at 1064 nm. The cavity mirror 25 has a high reflection coating for both infrared and visible, e.g. at 1064 nm and 532 nm. The output coupler 27 has a high transmission at 532 nm and a high reflection at 1064 nm. The laser cavity length is less than 60 cm for a short cavity decay time.

The pump head 60 consists of a solid-state gain medium of Nd:YAG laser rod. The laser rod is pumped with diode laser radiation delivered from multiple bars of high power diode lasers installed inside the pump head 60. The pump head 60 produces optical gain to generate fundamental laser radiation at 1064 nm. Other laser crystals such as Nd:YVO₄ may also be used for this application of soft tissue explosive ablation.

To obtain laser pulses with pulse width less than 150 ns and output peak power 60-200 kW at 532 nm, the Nd:YAG laser rod shall have a small diameter between 2 mm to 5 mm. Small laser rod diameter leads to higher single pass gain, which is necessary for short pulse generation. The rod is side pumped with diode laser radiation form three or more directions. Pumping the laser rod from three or more sides provides an angularly uniform gain distribution that leads to better beam quality and higher conversion efficiency. Other crystal shape and pumping configuration, such as slab or disk laser, may also produce desired laser power. For slab or disk laser, pump radiation from two directions is feasible.

The pump head 60 are preferably powered with a CW current. Pump head for high power diode-pumped laser is a delicate and expensive component. High power diode lasers have typically much longer lifetime to operate at CW mode than at pulsed mode. The cost is also substantially lower to operate high power diode lasers at CW mode than at pulsed mode.

Q-switch 49 and Q-switch 50 are acoustic-optical modulators with fast rise time. The Q-switches are operable at a repetition rate of 1 to 100 kHz. To enhance Q switching effect, Q-switch 49 and Q-switch 50 are installed with acousto-optical scattering directions perpendicular to each other. The two Q-switches are synchronized. The Q-switch rise time is less than 120 nanoseconds. Q-switch 50 is located near a cavity mirror 24 and Q-switch 49 is located on the other side of pump head 60.

The second harmonic generator 40 converts the fundamental laser radiation at 1064 nm into second harmonic laser radiation at 532 nm. The second harmonic generator 40 consists of a frequency-doubling crystal such as LBO or KTP and a temperature regulated device. The frequency-doubling crystal is mounted inside the temperature regulated device, which can control temperature electronically. LBO is more preferable for the diode-pumped laser because it has a power damage threshold at least 5 times higher than KTP. A temperature controller optimizes phase-matching conditions of the frequency-doubling crystal through temperature tuning. The crystal is fabricated for either type I phase-matching or type II phase-matching. Each end surface of the crystal has an anti-reflection coating for both infrared (1064 nm) and visible (532 nm) wavelength. The second harmonic generator 40 is placed near cavity mirror 25 where the intracavity beam 32 is substantially collimated with a small beam size, i.e. a beam waist. A small and collimated beam allows second harmonic generation more efficiently. The laser system 100 generates sub-150 ns pulses when the repetition rate is less than 15 kHz. Pulse energy up to 10 mJ is obtained and that leads to pulse fluence about 1000 mJ/cm² on target tissues.

As shown in FIG. 1, infrared laser beam path 30 is confined between cavity mirror 24 and cavity mirror 25, while visible laser beam path 32 is confined between cavity mirror 25 and output coupler 27. An output beam 33 with a wavelength at 532 nm exits from the output coupler 27. The output beam 33 is directed into a beam divergence controller 70 and then coupled into an optical fiber 80, of which a first end is mounted on a fiber mount 71. The beam divergence controller 70 and the fiber mount 71 form together a fiber coupler. A second end 90 of fiber 80 delivers the laser pulses, in cooperating with an endoscope or cystoscope, to target tissues, such as prostate tissues.

The laser controller 10 consists of a laser driver 61, a RF Q-switch driver 51, a first pulse compressor 52, an initial-inversion ratio controller 53, a pulse-repetition rate controller 54, a temperature controller 41, a temperature tuning circuit 42, and a primary cooling loop 21, and master control software 15. The laser controller 10 is connected to laser head 20 via an umbilical cable 11 and is powered via power cable 13.

The laser driver 61 provides DC current to power diode lasers installed inside the pump head 60. The laser driver 61 has control circuit to minimize amplitude fluctuation of the laser pulses via current control or power control the pump radiation from the diode lasers. Pulse fluctuation can be suppressed to below 10%, typically 1%, to achieve smoother tissue ablation.

The initial-inversion ratio controller 53 controls the duty circle of the Q-switching gate signal. The pulse repetition rate controller 54 controls the laser pulse repetition rate. To achieve optimal operation, the master control software 15 optimizes the initial-inversion ratio controller 53 and the pulse repetition rate controller 54. The RF Q-switch driver 51 provides RF power to drive Q-switch 49 and Q-switch 50 simultaneously. The First pulse compressor 52 compresses the possible giant laser optical pulse when switching between operation modes. The temperature controller 41 maintains a stable temperature for the second harmonic generator 40. Another function of the temperature controller 41 is to optimize phase matching conditions under different operation conditions. The primary cooling loop 21 provides direct water-cooling of the laser head 20. The primary cooling loop 21 removes heat generated inside the pump head 60, the RF Q-switches 49 and 50, and the second harmonic generator 40.

In the first embodiment, the small diameter laser rod makes a good contribution to short pulse width and high peak power because of higher single pass gain. Dual Q-switch configuration further shortens the laser pulses with enhanced Q switch action. Short cavity length contributes to pulse shortening via a short cavity decay time. The control electronics, including the initial-inversion ratio controller 53 and the pulse repetition rate controller 54, optimizes the laser performance by balancing the pulse duration, pulse peak power, and laser average power. All combined efforts enable the laser system to produce sub-150 ns pulses with higher than 60 kW peak power at 532 nm.

It is well known in the art that water-cooling of a high power laser is always a delicate and challenging task, requiring fast flow of distilled or deionized water. A primary cooling loop is typically a closed loop cooling system, enabling the use of distilled or deionized water. When power consumption is within a few kilowatts, heat removed by primary cooling loop may dissipate through airflow. When power consumption is more than a few kilowatts, external water-cooling or a second cooling loop is a common solution to carry the heat out of the laser room.

The infrared folding mirror 26 and the output coupler 27 are spherical mirror to accommodate and control the cavity mode, in cooperating with thermal lens induced inside the laser rod located in the pump head 60. The number of transverse modes to operate can be determined by cavity design. The number of transverse modes is typically much bigger than 10 for optimal diode pumping efficiency. In the embodiment of FIG. 1, the diode-pumped laser operated at highly multiple modes can have a conversion efficiency of about 10% from the pump diode laser radiation. Conversion efficiency may reduce to below 2% when operation is limited toward single transverse mode, i.e. TEM₀₀ mode. Thus, multiple mode operation shall easily lead to a conversion efficiency of 3% or higher from diode laser radiation.

Another delicate and challenging work with the diode-pumped laser is to protect the laser crystal and frequency-doubling crystal from power damage. Operating the high power diode-pumped laser system 100 at highly multiple modes is a first measure to protect the crystals from power damage. Multiple-modes operation generates a much bigger beam size in either the laser crystal or the frequency doubling crystal and thus reduces the peak power density inside the crystals. Beam waist of this laser system 100 is about 3 or 5 times as big as what would be for a TEM₀₀ mode.

A second measure implemented to protect the crystals is to compress the giant first-pulse of each Q-switched pulse train when switching between high peak power operation and low peak power operation. The first-pulse compressor 52 is used to incorporate with the Q-switch driver 51 and to modify the transient Q value of laser cavity to eliminate any giant first-pulses. One way to implement first-pulse compressor 52 is an electronic circuit to modify transient RF power applied to the Q-switches 49 and 50.

A third measure implemented to protect the frequency-doubling crystal is to tune crystal temperature slowly via a temperature tuning circuit 42. The second harmonic generator 40 is temperature regulated by a temperature controller 41 to maintain stable phase matching angle. Phase matching angle is a variable of temperature and mechanical position. Mechanical or temperature drift may deviate the phase matching condition from perfect. The temperature tuning circuit 42 is to tune and to compensate phase mismatching due to environment temperature and/or mechanical drift to optimal phase matching angle. Mechanical tuning might cause a temperature shock inside the frequency-doubling crystal due to a sudden change in optical path or heat loading. Such a temperature shock would damage the crystal easily. Temperature tuning can eliminate the need to open laser cover during angle adjustment, thus further reducing possible human mistakes and improving laser reliability.

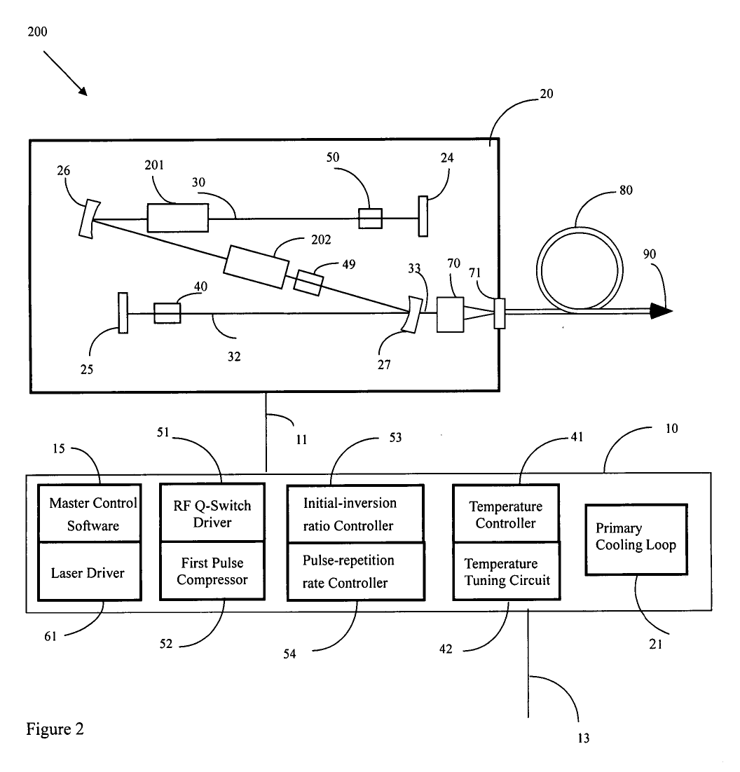

FIG. 2 is a schematic diagram showing a second embodiment of a diode-pumped laser system 200 for soft tissue explosive ablation. Two smaller diameter pump heads 201 and 202 are arranged in cascade to replace the pump head 60 in laser system 100. In this configuration, the laser cavity has a higher single pass gain. Laser rod of Nd:YAG or Nd:YVO₄ shall have a small diameter between 2 mm to 4 mm, preferably 3 mm. The laser system 200 has demonstrated laser pulses with pulse duration shorter than 40 ns and peak power higher than 100 kW, at a repetition rate up to 15 kHz.

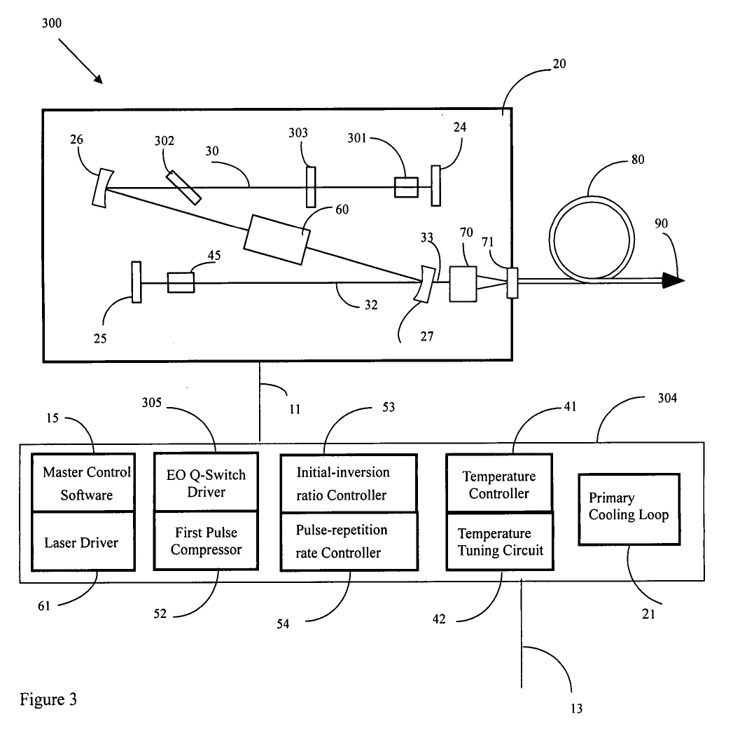

FIG. 3 is a schematic diagram showing a third embodiment of a diode-pumped laser system 300 for soft tissue explosive ablation. An electro-optical Q-switch 301 is used to replace AO Q-switches 49 and 50. An EO Q-switch driver 305 of laser controller 304 replaces the RF driver 51 in laser controller 10. Electro-optical Q-switching requires intracavity near-infrared beam 30 to be linearly polarized. A Brewster angle polarizer 302 is used to force the intracavity beam 30 being in linear polarization. When EO Q-switch driver 305 sends a driving signal to OE Q-switch 301, it rotates beam 30 polarization by 45 degrees in a single pass or 90 degrees in a double pass. After passing a quarter-wave plate 303, the intracavity beam 30 can pass the Brewster polarizer 302 without attenuation. After the driving signal, OE Q-switch 301 does not rotate beam 30 polarization, and the quarter-wave plate 303 and the Brewster polarizer 302 together introduce a big cavity loss to stop lasing. Because EO Q-switch has very fast modulation, laser system 300 is capable to produce laser pulses with sub-10 ns duration and 200 kW peak power at 10 kHz repetition rate. In this embodiment, the nonlinear crystal is fabricated for type I phase-matching for second harmonic generation.

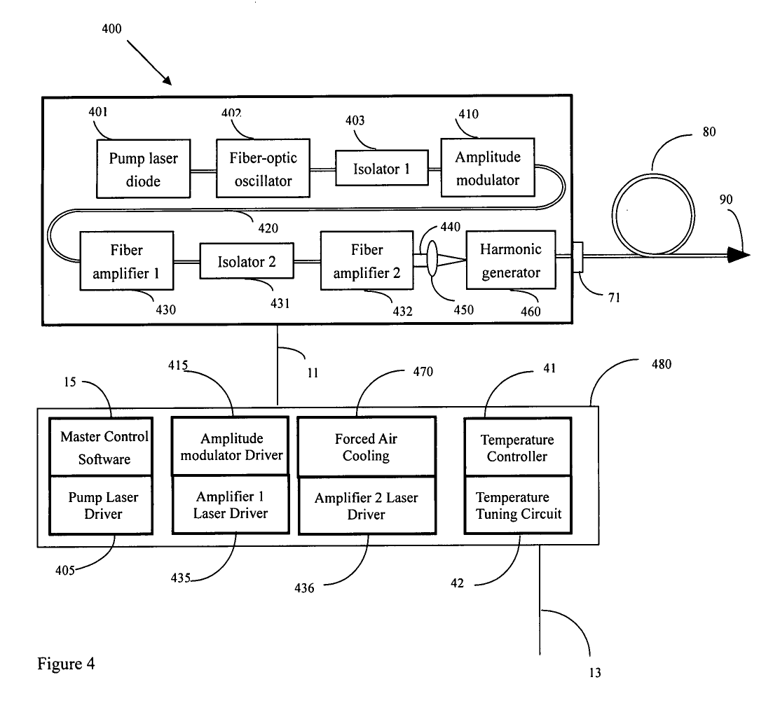

FIG. 4 is a schematic diagram showing a fourth embodiment of a diode-pumped fiber laser system for soft tissue explosive ablation. The laser system 400 is a large core Yb-doped fiber laser, frequency-doubled to produce laser pulses at 540 nm. The laser system 400 consists of a fiber optic oscillator 402, which is powered by pump laser diode 401. The pump laser diode 401 and fiber oscillator 402 form a master oscillator producing low power laser light around 1080 nm. There are at least two fiber Bragg gratings inside the master oscillator to narrow down the laser line-width. Narrow laser line-width is required for efficient frequency doubling by nonlinear crystals such as LBO or KTP. The fiber isolator 1 403 is to isolate and protect the fiber optic oscillator 402 from undesired feedback to the oscillator 402. Amplitude modulator 410 is a high frequency acousto-optical modulator, which is commonly used in optical telecommunications. The amplitude modulator 410 is powered and controlled by amplitude modulator driver 415. The amplitude modulator 410 choppers the laser beam generated into a train of pulses with repetition rate of kilohertz to megahertz and pulse width of 1-5 ns. The short laser pulses propagate through fiber 420 to the first stage fiber amplifier 1 430. After first stage amplification, the laser pulses enter isolator 2 431, then the amplifier 2 432 for power amplification. Both amplifier 1 430 and amplifier 2 432 are large core Yb-doped amplifiers, which each is pumped by diode lasers. The amplifier 1 430 and amplifier 2 432 are driven by amplifier 1 laser driver 435 and amplifier 2 laser driver 436, respectively. A fiber-oscillator-power-amplifier (FOPA) is formed with the pump laser diode 401, fiber optic oscillator 402, isolator 1 403, amplitude modulator 410, transmission fiber 420, fiber amplifier 1 430, isolator 2 431, and fiber amplifier 2 432. Having two stages of amplification, the amplified laser pulses can have a peak power more than 40 kW. A focal lens 450 focuses the amplified laser beam 440 onto an external cavity harmonic generator 460 to produce frequency doubling.

The second harmonic generator 460 consists of a frequency-doubling crystal, such as LBO or KTP, and a temperature regulated device. The frequency-doubling crystal is mounted inside the temperature-regulated device. LBO is more preferable for high peak power laser because it has a power damage threshold at least 5 times higher than KTP. The crystal is fabricated for either type I phase-matching or type II phase-matching. Each end surface of the crystal has an anti-reflection coating for both infrared (1080 nm) and visible (540 nm) wavelength. In order to achieve the highest possible frequency-doubling efficiency, the second harmonic generator 460 can consist of two or more nonlinear crystals in single-pass configuration or one nonlinear crystal in double-pass configuration. The final green beam enters the fiber coupler 71 coupling to fiber 80. The peak power at exit end 90 of fiber 80 is over 20 kW. This fiber laser system 400 has diffraction-limited beam quality and can produce a peak power density more than 10 MW/cm² on target tissues.

This laser system 400 has very high electrical conversion efficiency, about 10% from input electrical power to optical power output. The high conversion efficiency reduces cooling requirement, and air-cooling is sufficient. The laser controller 480 is equipped with forced air-cooling 470.

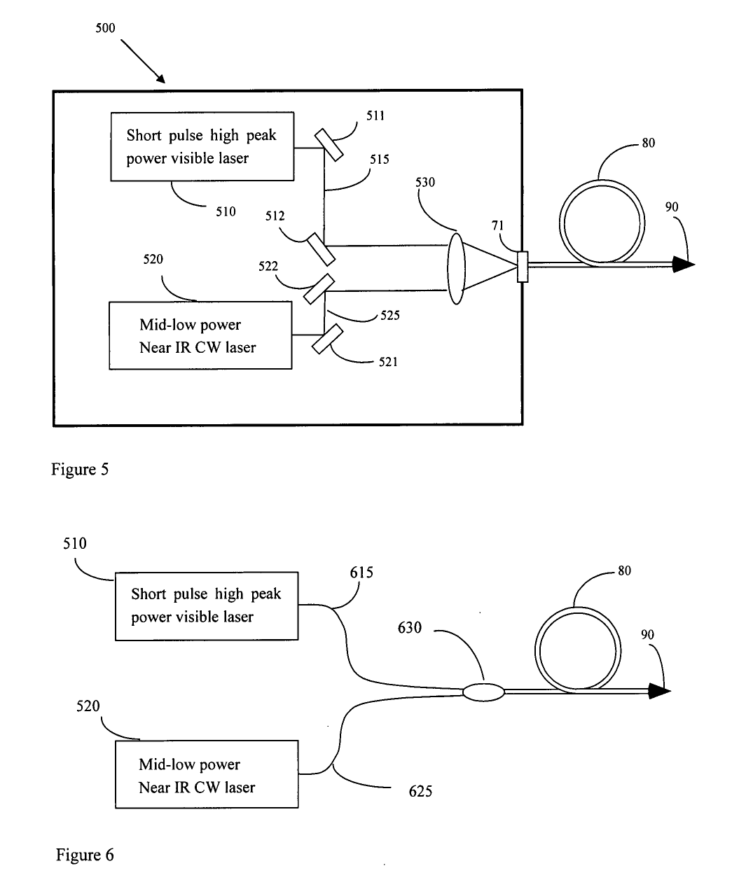

FIG. 5 is a schematic diagram showing a fifth embodiment. Laser system 500 is a dual-wavelength surgical laser system consisting of a laser 510 for tissue explosive ablation and a laser 520 for tissue coagulation. The output beam 515 from the laser 510 propagates in free space and is guided by tuning mirrors 511 and 512 to fiber coupling lens 530. The output beam 525 from the laser 520 propagates in free space and is guided by tuning mirrors 521 and 522 to focal lens 530. After focal lens 530, the beam 515 and beam 525 are both coupled into fiber coupler 71, enter fiber 80, and exit at exit 90. The laser 510 can be either laser system 100, 200, 300, or 400. The laser 520 can be continuous wave 1064 nm Nd:YAG laser, 1064 nm Nd:YVO4 laser, 800-1000 nm diode laser, 800-100 nm fiber-coupled diode laser, or 1000-1400 nm fiber laser. For best hemostasis, the laser 520 shall have 5-30 W average power on target tissues.

FIG. 6 is a schematic diagram showing anther laser beam coupling of the fifth embodiment. The laser output from the laser 510 propagates in fiber 615. The laser output from the laser 520 propagates in fiber 625. Lasers in fiber 615 and fiber 625 are coupled to a fiber 80 via fiber combiner 630. The laser light finally exits at exit 90. Because LITEA may have a thinner coagulation layer, the dual-wavelength surgical laser systems of FIG. 5 and FIG. 6 are useful for hemostatic surgery.

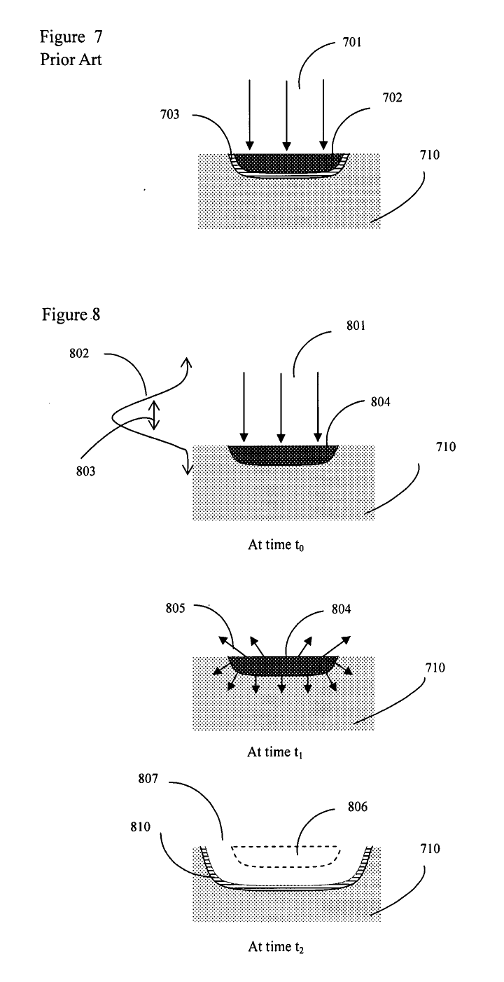

FIG. 7 illustrates photoselective tissue vaporization of prior art. Oxyhemoglobin has a strong optical absorption for 532 nm green laser light. Fresh soft tissue 710, such as fresh prostate tissue, has high concentration of oxyhemoglobin. When average power density is higher than 10 kW/cm², visible laser 701 irradiates on fresh soft tissue 710, vaporizes a volume 702, and leaves a coagulation layer 703. The primary chromophore for laser absorption is oxyhemoglobin. The laser-tissue interaction is named photoselective.

FIG. 8 illustrates a thermal explosive ablation of fresh tissue 710. At 532 nm wavelength, both oxyhemoglobin (HbO₂) and deoxyhemoglobin (Hb) have similar optical absorption coefficient. The optical absorption coefficient μ_{a(fresh tissue)} of fresh soft tissue 710, such as fresh prostate tissue, is determined by the hemoglobin μ_{a (hemoglobin)}. The μ_{a(fresh tissue)} is about 200-300 cm⁻¹, depending on actual blood vessel distribution. Consider that a laser pulse 801, having a short pulse width and a high peak power at 532 nm, irradiates on tissue 710. The laser pulse has temporal profile 802 and pulse width tₚ 803. At the end of pulse duration tₚ 803, i.e., at time t₀, most of laser energy is absorbed in a volume 804 with a thickness of 1/μ_{a(fresh tissue)}, approximately 30-50 micros. For most soft tissues, including hemoglobin, the thermal diffusivity is between 0.5×10⁻⁷ m²/s to 1.5×10⁻⁷ m²/s. It can be calculated that a sub-150 ns laser pulse shall generate thermal confinement inside the tissue volume 804. Since blood and water are major liquid in fresh prostate tissue, the speed of sound ca is about 1.5×10³ m/s. It can be calculated that a sub-30 ns laser pulse can also generate stress confinement inside the tissue volume 804. At time t₁, right after laser illumination, the irradiated tissue volume 804 has been heated to superheat temperature, usually between 300-400° C., and starts an explosion 805. If the laser pulse width is less than 30 ns, i.e., both thermal and stress confinements are met, the explosion 805 will have a very violent material ejection. Due to thermal explosive effect, at end of laser ablation time t₂, the ablated cavity 807 has a bigger volume than the irradiated tissue volume 804 or the empty volume 806. After ablation, there is a layer of coagulated tissue 808 formed at the boundary of ablated cavity 807. Because of the thermal confinement, thickness of coagulation layer 808 is less than 1 mm. The laser systems of the first and second embodiments can produce thermal confined laser ablation, while the laser systems of the third and fourth embodiments are capable to produce thermal and stress confined explosive ablation. In FIG. 8, the optical absorption chromophore is hemoglobin and the laser absorption is photoselective. The ablation efficiency, however, is significantly improved if pulse duration tₚ 803 is shorter than 150 ns and is further improved if pulse duration tₚ 803 is shorter than 30 ns.

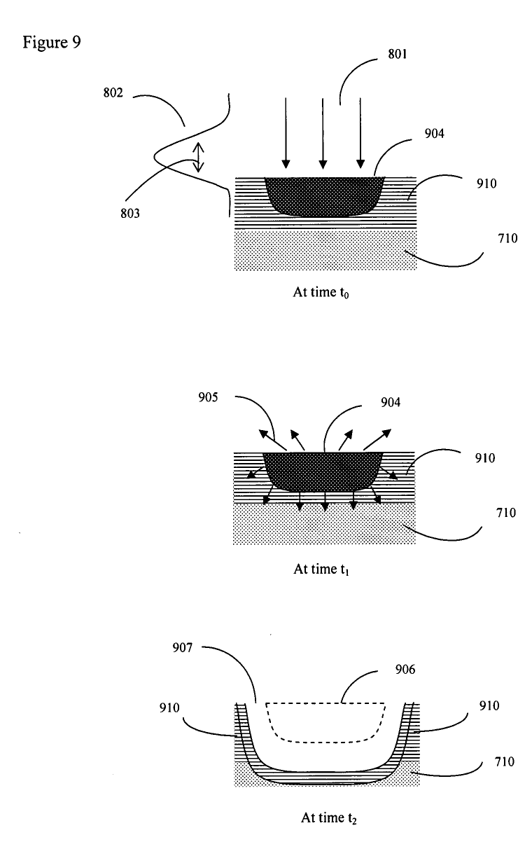

FIG. 9 illustrates a thermal explosive ablation of coagulated tissue 910. In laser ablation surgery, such as laser ablation prostatectomy, tissue coagulation is inherent. After an initial laser ablation of fresh tissue, a layer of coagulated tissue 910 is formed. The coagulation layer 910 has a thickness between 0.5 to 2 mm, depending on how strong the thermal confinement is and how many laser pulses continuously applied on the same area. Underneath the coagulation layer 910 is a thermally untouched fresh tissue 710. Because hemoglobin concentration inside coagulated tissue 910 is very minimal, optical absorption coefficient μ_{a(coagulated tissue)} of coagulated layer is about 10-40 cm⁻¹ at 532 nm. The actual value depends on the degrees of coagulation and tissue structure. In order to remove tissues effectively, laser peak power density on the coagulated tissue shall be high enough to produce both thermal and stress confinements. Consider that a laser pulse 801, having a short pulse width and high peak power at 532 nm, irradiates on coagulated tissue layer 910. The laser pulse has a pulse temporal profile 802 and pulse width tₚ 803. At the end of pulse duration tₚ 803, i.e., at time t₀, most of laser energy is absorbed in a volume 904 with a thickness of 1/μ_{a(coagulated tissue)}, approximately 250-1000 micros. The thermal diffusivity of coagulated tissue 910 has an order of 1.5×10⁻⁷ m²/s. It can be calculated that a sub-150 ns laser pulse definitely generates thermal confinement inside the tissue volume 904 before time t₁ when the laser pulse finishes the irradiation. At time t₁, the irradiated tissue volume 904 has been heated to a superheat temperature, usually between 300-400° C., and starts an explosion 905. In coagulated tissue, water is the major liquid for heat dissipation. It is known that the speed of sound cₐ inside coagulated tissue is about 1.54×10⁻³ m/s. It can be calculated that a sub-160 ns laser pulse can also generate stress confinement inside the tissue volume 904. Any laser system of the embodiments is capable to produce sub-150 ns pulses with high peak power for thermal and stress confinement at time t₀. Therefore, the explosion 905 at time t₁ shall have violent material ejection. Due to thermal explosive effect, at end of laser ablation time t₂, the ablated cavity 907 has a bigger volume than the irradiated tissue volume 904 or empty volume 906. After ablation, there is a layer of coagulated tissue 910 formed at the boundary of the ablated cavity 907. Because of the thermal confinement, thickness of the new coagulation layer 910 is between 0.5-1 mm from the fresh tissue 710. Because stress confinement is also satisfied, any laser system of the embodiments can produce efficient laser explosive ablation.

In FIG. 9, the primary optical absorption chromophore is the coagulated soft tissue, e.g., coagulated prostate tissue. The laser-tissue interaction is thermal and stress confined explosive ablation. The microchomophore centers are coagulated tissues, whose volume is substantially smaller than the volume of the total coagulated tissues 904. Laser pulse with short pulse width and high peak power induces superheating of these microchomophore centers under conditions of thermal confinement and stress confinement. The overheated microchomophore centers go to explosive boiling and remove mechanically the entire irradiated tissue bulk 907, which is bigger than irradiated coagulation tissue volume 904.

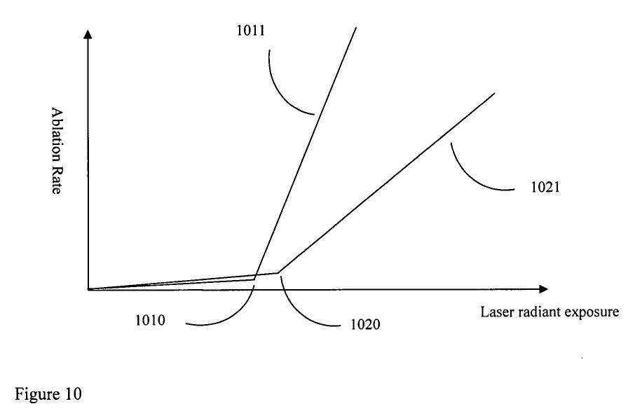

FIG. 10 illustrates a thermal explosive tissue ablation rate depends on laser pulse duration. Under stress confinement, the peak stress increases as the laser pulse width getting shorter. High peak stress results in low ablation threshold and high mass removal rate m. Ablation threshold 1010 of short pulse is lower than ablation threshold 1020 of long pulse; and ablation rate 1011 of short pulse is steeper than ablation rate 1021 of long pulse. Threshold fluence is estimated to be 100 mJ/cm² or higher, depending on pulse duration and tissue absorption.

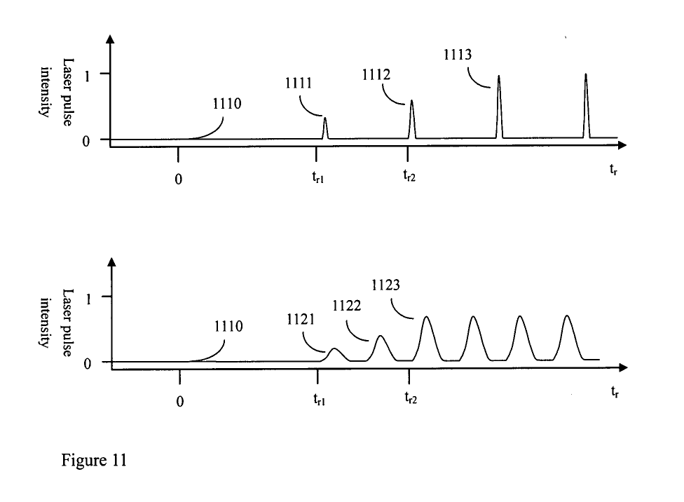

FIG. 11 illustrates peak power reduction when pulse repetition rate increases. When coagulation is needed for hemostasis, peak power of the pulses can be reduced via reducing Q-switch duty cycle or pulse repetition rate. By doing so, the laser pulse width stretches to a several hundred nanoseconds or so, while laser peak power density drops to below 1 MW/cm² on target tissue surface. Pulses 1111, 1112, and 1113 are more desirable for thermal explosive ablation, while pulses 1121, 1122, and 1123 are more desirable for tissue coagulation. Due to a mechanism of first pulse compression, pulses 1111 and 1112 have lower amplitude or peak power than normal pulse 1113. The ablation pulses have shorter pulse width, higher peak power and lower pulse repetition rate. In contrast, the coagulation pulses have longer pulse width, lower peak power and higher pulse repetition rate.

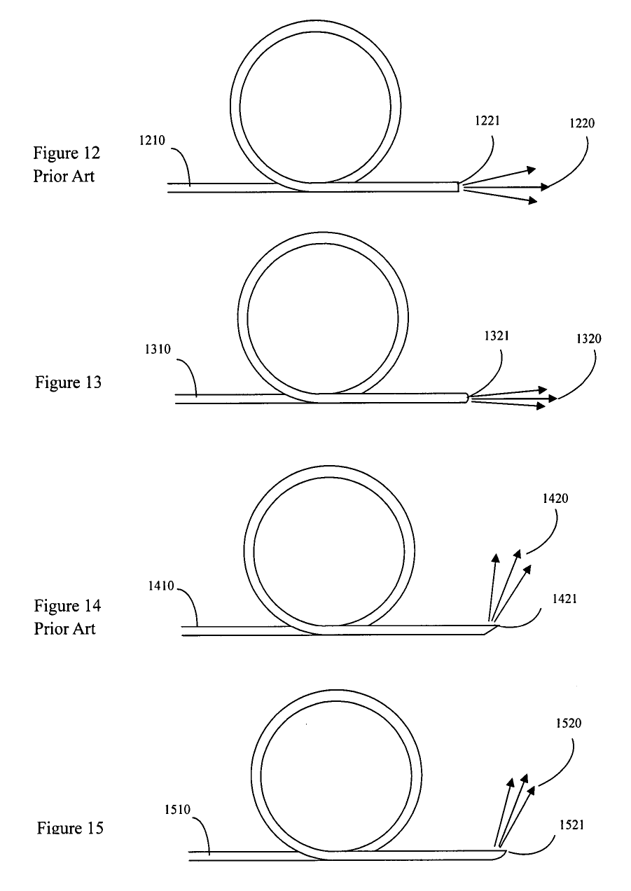

FIG. 12 illustrates a prior art fiber tip 1221 of optical fiber 1210. The facet of the fiber tip 1221 is a flat polished surface, and output beam 1220 is a divergent beam. The divergent angle of the beam depends on the beam quality, as well as numerical aperture of the optical fiber 1210. For a large core fiber, such as the 600-micro fiber used in prior art laser ablation prostatectomy, laser beam quality is attributed to an undesirably big divergent angle.

FIG. 13 illustrates a modified fiber tip 1321 of optical fiber 1310. The facet of the fiber tip 1321 is a curved surface, and output beam 1320 is a less divergent beam, e.g. an NA of 0.22 or smaller. A less divergent output beam 1320 shall have a smaller beam spot and thus a peak higher power density on target tissue, in comparing to an otherwise divergent beam 1220. A less divergent output beam 1320 shall also lead to a slower power density change with respect to distance between the fiber tip 1321 and the target tissue, which allows a surgeon to control more easily laser ablation on target tissue.

FIG. 14 illustrates another prior art fiber tip 1421 of optical fiber 1410. The facet of the fiber tip 1421 is a flat but inclined surface, and output beam 1420 is a divergent beam deflected to one side of the optical fiber 1410.

FIG. 15 illustrates another modified fiber tip 1521 of optical fiber 1510. The facet of the fiber tip 1521 is a curved and inclined surface, and output beam 1520 is a less divergent beam deflected to one side of the optical fiber 1510. A less divergent output beam 1520 shall have a smaller beam spot and thus a higher peak power density on target tissue, in comparison with an otherwise divergent beam 1420. A less divergent output beam 1520 shall also lead to a slower power density change with respect to distance between the fiber tip 1521 and target tissue aside from the tip, which shall allow a surgeon to control more easily laser ablation on target tissue aside form the fiber 1510.

Although the above description is based on preferred embodiments to illustrate the present invention, various modifications can be made without departing from the scopes of the appended claims.

What is claimed is:

1. A surgical apparatus for laser ablation of soft tissue, comprising:
a diode-pumped laser pumped with CW diode-laser radiation and operated at highly multiple transverse modes;
a Q-switch modulating said laser to produce laser pulses with pulse repetition of 1 kHz or higher and pulse energy of 1 mJ or higher;
short-pulse means shortening said laser pulses to 300 ns or shorter; and
laser-delivery means delivering said laser pulses onto said soft tissue and producing a pulse fluence of 100 mJ/cm² or higher;
wherein said surgical apparatus produces laser-induced thermal explosive ablation on said soft tissue.

2. A surgical apparatus of claim 1, further comprising:
a pulse stabilizer incorporated into said laser to minimize amplitude fluctuation of said laser pulses to 10% or lower;
wherein said surgical apparatus produces a smooth laser ablation on said soft tissue.

3. A surgical apparatus of claim 1, further comprising:
dual-wavelength means incorporated into said laser to obtain a predetermined ratio of pulse energy in visible spectrum and pulse energy in infrared spectrum;
wherein said surgical apparatus creates a coagulation layer for hemostasis.

4. A surgical apparatus of claim 1 wherein said soft tissue includes prostate tissue.

5. A surgical apparatus of claim 1 wherein said diode-pumped laser includes:
a solid-state gain medium having a cross-section dimension of 2 mm or bigger to support operation of said highly multiple transverse modes; and
diode laser radiation delivered from multiple bars of diode lasers and pumping said solid-state gain medium from three or more directions;
wherein a beam spot size at said solid-state gain medium is at least three times as big as its minimum, which is associated with TEM00 mode operation.

6. A surgical apparatus of claim 1 wherein said Q-switch is operated at a quasi-CW mode.

7. A surgical apparatus of claim 1 wherein said short-pulse means utilizes 2 AO Q-switches with crossed polarizations.

8. A surgical apparatus of claim 1 wherein said short-pulse means utilizes an EO Q-switch.

9. A surgical apparatus of claim 1 wherein short-pulse means utilizes 2 laser rods.

10. A surgical apparatus of claim 1 wherein said diode-pumped laser includes a LBO crystal for 2^{nd} harmonics generation.

11. A surgical apparatus of claim 1 wherein said diode-pumped laser operates at a green spectrum.

12. A surgical apparatus of claim 1 wherein said laser pulses have a repetition rate in a range of 10 kHz to 25 kHz.

13. A surgical apparatus of claim 1 wherein said laser pulses has pulse duration in a range of 40 ns to 150 ns.

14. A surgical apparatus of claim 1 wherein said laser pulses has pulse energy in a range of 2.5 to 15 mJ.

15. A surgical apparatus of claim 1 wherein said pulse fluence on said soft tissue is in a range of 250 mJ/ cm² to 1000 mJ/ cm².

16. A surgical apparatus of claim 1 wherein said optical means delivers said laser pulses onto said soft tissue with an exit NA of 0.22 or smaller.

17. A surgical apparatus for laser ablation of prostate tissue, comprising:
a diode-pumped laser pumped with CW diode-laser radiation and operated at highly multiple transverse modes;
a Q-switch operated at a quasi-CW mode to produce laser pulses with pulse repetition of 1 kHz or higher and pulse energy of 1 mJ or higher;
short-pulse means shortening said laser pulses to 300 ns or shorter;
a LBO crystal producing 2^{nd} harmonics generation to a green wavelength; and
laser-delivery means delivering said laser pulses onto said soft tissue and producing a pulse fluence of 100 mJ/cm² or higher;
wherein said surgical apparatus produces laser-induced thermal explosive ablation on said prostate tissue.

18. A surgical apparatus of claim 17, further comprising:
a pulse stabilizer incorporated into said laser to minimize amplitude fluctuation of said laser pulses to 10% or lower;
wherein said surgical apparatus produces a smooth laser ablation on said soft tissue.

19. A surgical apparatus of claim 17, further comprising:
dual-wavelength means incorporated into said laser to obtain a predetermined ratio of pulse energy at green wavelength and pulse energy at infrared wavelength;
wherein said surgical apparatus creates a coagulation layer for hemostasis.

20. A surgical method for laser ablation of soft tissue, comprising the steps of:
operating a diode-pumped laser at highly multiple transverse modes;
pumping said laser with CW diode-laser radiation;
modulating said laser to produce laser pulses with pulse repetition of 1 kHz or higher and pulse energy of 1 mJ or higher;
shortening said laser pulses to 300 ns or shorter; and
delivering said laser pulses onto said soft tissue to produce a pulse fluence of 100 mJ/cm² or higher;
wherein said surgical apparatus produces laser-induced thermal explosive ablation on said soft tissue.

21. A surgical apparatus for laser ablation of soft tissue, comprising:
a diode-pumped laser pumped with CW diode-laser radiation and coupled into optical fiber;
a modular means modulating said laser to produce laser pulses with pulse repetition of 1 kHz or higher and pulse duration of 300 ns or shorter;
amplifier means amplifying said laser pulses to a peak power of 1 kW or higher; and
laser-delivery means delivering said laser pulses onto said soft tissue and producing a pulse fluence of 100 mJ/cm² or higher;
wherein said surgical apparatus produces laser-induced thermal explosive ablation on said soft tissue.

22. A surgical apparatus of claim 21 wherein said optical filer is doped with ytterbium (Yb) or thulium (Tm).

23. A surgical apparatus of claim 21 wherein said amplifier means employs one or more stages of fiber amplifier.

24. A surgical apparatus of claim 21, further comprising a harmonic generator to convert the fundamental wavelength to wavelengths suitable for soft issue optical absorption.
